(19) United States
(12) Patent Application Publication
(10) Pub. No.: US 2009/0088679 A1
(43) Pub. Date: Apr. 2, 2009

(54) Electronically-Degradable Layer-by-Layer Thin Films

(75) Inventors: Kris Wood, Cambridge, MA (US); Nicole Zacharia, Hinsdale, IL (US); Paula Hammond Cunningham, Newton, MA (US); Dean Delongchamp, Gaithersburg, MD (US)

Correspondence Address: CHOATE, HALL & STEWART LLP, TWO INTERNATIONAL PLACE, BOSTON, MA, 02110, US

(73) Assignee: MASSACHUSETTS INSTITUTE OF TECHNOLOGY, Cambridge, MA (US)

(21) Appl. No.: 11/815,718

(22) PCT Filed: Feb. 7, 2006
(86) PCT No.: PCT/US06/04295

§ 371 Date: Oct. 29, 2008

Related U.S. Application Data
(60) Provisional application No. 60/650,613, filed on Feb. 7, 2005.

(51) Int. Cl. A61N 1/30; A61K 9/70

(52) U.S. Cl. 604/20; 424/443

(57) ABSTRACT

A decomposable thin film comprising a plurality of alternating layers of net positive and negative charge. At least a portion of the positive layers, the negative layers, or both, comprise a polyelectrolyte.

This application claims the priority of U.S. Provisional Application No. 60/650,613, filed Feb. 7, 2005, the contents of which are incorporated by reference herein.

### FIELD OF THE INVENTION

This invention relates to layer-by-layer thin films that may be degraded by application of an electrical voltage.

### BACKGROUND OF THE INVENTION

The ability to deliver multiple doses of drug in precise quantities to the body in a pre-programmed manner is highly desirable for a number of therapeutic applications. In particular, the regular delivery of toxic cancer drugs, potent therapeutics, anaesthetics or other agents to hard-to-reach regions of the body, such as the brain, is considered one of the most difficult challenges in the world of drug delivery. Many regimens require the repeated administration of the drug via oral intake or syringe injection at or near the desired site, thus requiring costly monitoring, the risk of missed dosages, and for delivery to difficult regions such as the brain or the excavation site of a tumor, administration of medication may even require multiple surgeries. A recent area of great interest is the use of medical implants as a means of delivering drugs. In such cases, the drug can be incorporated into a film or plastic matrix, upon which it undergoes slow diffusion or dissolution to free the drug over sustained periods [1]. Thus far, the primary commercial example of a drug release implant coating is that used for stents in arterial applications [2-5]. This method has many advantages, but lacks the ability to control the administration of doses on a fine level; more specifically, pulsatile or periodic fluctuations of drug level are sometimes desired for a given drug application, but such release profiles cannot be replicated by traditional coatings. A second desired advantage would be the ability to control and alter the amount of drug released and the release profile after insertion of the implant, thus allowing adjustments in drug level that depend on the condition of the patient. This capability can ultimately lead to fully responsive drug systems that increase levels of a drug with a prescribed physiological change.

To achieve these advantages, microfluidic devices and sensors have been microfabricated and used as implants that can deliver varying amounts of drug [7-10]. A more general delivery approach has been demonstrated in which microwells are created in a silicon or silicon nitride substrate, filled with different drugs, and then coated with a thin layer of gold which acts as a capping layer to retain the drug in the well [11,7]. When individual wells are addressed with a low electrochemical potential, the thin gold film dissolves, and the drug in the well is released as a singular pulse. This approach has led to significant change in the way drug delivery is viewed—it is now possible to create fairly complex drug release profiles by directly addressing different wells in the grid.

Several challenges remain in achieving highly controlled drug release from implant devices. One issue is the integration of this level of control on nonplanar, functional or structural implants such as arterial stents, medical sutures, bone implants, tissue replacements, etc. A means of creating a conformal thin film coating on nonplanar, and in some cases flexible surfaces, which can undergo remotely controlled and variable dissolution to release a complex drug profile would be of extreme interest in such applications, particularly if such coatings were inexpensive and easily processed. A second challenge involves the ultimate limits in the quantity of drug that can be delivered using microwell technologies; unless a drug reservoir is used in these techniques, the amount delivered is limited to small volumes determined by the well size or channel dimensions in microfluidic applications. A third challenge involves the potential simplification of microchip designs utilizing soft lithography and thin film approaches rather than the more expensive and extensive micromachining and drug loading steps. The opportunity to incorporate more complex drug release profiles within singular thin films would lead to individualized dosages of multiple drugs on a chip, thus making the use of multiple wells for a given drug release profile unnecessary.

### DEFINITIONS

“Biomolecules”: The term “biomolecules”, as used herein, refers to molecules (e.g., proteins, amino acids, peptides, polynucleotides, nucleotides, carbohydrates, sugars, lipids, nucleoproteins, glycoproteins, lipoproteins, steroids, etc.) whether naturally-occurring or artificially created (e.g., by synthetic or recombinant methods) that are commonly found in cells and tissues. Specific classes of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

“Polyelectrolyte” or “polyion”: The terms “polyelectrolyte” or “polyion”, as used herein, refer to a polymer which under some set of conditions (e.g., physiological conditions) has a net positive or negative charge. Polycations have a net positive charge and polyanions have a net negative charge. The net charge of a given polyelectrolyte or polyion may depend on the surrounding chemical conditions, e.g., on the pH.

“Polynucleotide”, “nucleic acid”, or “oligonucleotide”: The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least three nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages). The polymer may also be a short strand of nucleic acids such as siRNA.

“Polypeptide”, “peptide”, or “protein”: According to the present invention, a “polypeptide”, “peptide”, or “protein” comprises a string of at least three amino acids linked together by peptide bonds. The terms “polypeptide”, “peptide”, and “protein”, may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain; see, for example, http://www.cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polysaccharide”, “carbohydrate” or “oligosaccharide”: The terms “polysaccharide”, “carbohydrate”, or “oligosaccharide” refer to a polymer of sugars. The terms “polysaccharide”, “carbohydrate”, and “oligosaccharide”, may be used interchangeably. Typically, a polysaccharide comprises at least three sugars. The polymer may include natural sugars (e.g., glucose, fructose, galactose, mannose, arabinose, ribose, and xylose) and/or modified sugars (e.g., 2′-fluororibose, 2′-deoxyribose, and hexose).

“Small molecule”: As used herein, the term “small molecule” is used to refer to molecules, whether naturally-occurring or artificially created (e.g., via chemical synthesis), that have a relatively low molecular weight. Typically, small molecules are monomeric and have a molecular weight of less than about 1500 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§ 330.5, 331 through 361, and 440 through 460; drugs for veterinary use listed by the FDA under 21 C.F.R. §§ 500 through 589, incorporated herein by reference, are all considered acceptable for use in accordance with the present invention.

“Bioactive agents”: As used herein, “bioactive agents” is used to refer to compounds or entities that alter, inhibit, activate, or otherwise affect biological or chemical events. For example, bioactive agents may include, but are not limited to, anti-AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, and imaging agents. In certain embodiments, the bioactive agent is a drug.

A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, and the United States Pharmacopeia-25/National Formulary-20, published by the United States Pharmcopeial Convention, Inc., Rockville Md., 2001, all of which are incorporated herein by reference.

### SUMMARY OF THE INVENTION

In one aspect, the invention is a decomposable thin film including a plurality of alternating layers of net positive and negative charge. At least a portion of the positive layers, the negative layers, or both, include a polyelectrolyte. The layers are stable with respect to delamination at a first predetermined voltage and the thin film is not stable at a second predetermined voltage. The first predetermined voltage may be no applied voltage. At least a portion of the layers of net positive charge may include a first polyelectrolyte that carries a positive charge at the first predetermined voltage. The first polyelectrolyte may included a polymer having ionizable groups selected from amine, quaternary ammonium, quaternary phosphonium, and any combination of these, which ionizable groups may be disposed in groups pendant from a backbone of the polymer, attached to the backbone directly, or incorporated in the backbone of the polymer.

At least a portion of the layers of net negative charge may include a second polyelectrolyte that carries a negative charge at the first predetermined voltage. The second polyelectrolyte many include a polymer having ionizable groups selected from carboxylate, sulfonate, sulfate, phosphate, nitrate, and combinations of the above, which ionizable groups may be disposed in groups pendant from a backbone of the polymer, attached to the backbone directly, or incorporated in the backbone of the polymer.

At least a portion of the layers may include a conducting polymer, a redox polymer, or a dendrimer. At least a portion of the layers of net negative charge may include Prussian Blue. At least a portion of the layers may include a first active agent, for example, a drug, a protein, an oligopeptide, or an polynucleotide. The first active agent may be encapsulated by a micelle, a dendrimer, or a nanopartical. The first active agent may be retained on the polyoelectrolyte in the positive or negative layers by covalent or non-covalent interactions. The concentration of the first active agent may vary among the layers. For example, the concentration may describe a gradient from a top layer of the film to a bottom layer of the film. The thin film may include alternating pluralities of layers that do and do not contain the first active agent. At least a portion of the layers may include a second active agent, both the first active agent and the second active agent, or either the first active agent or the second active agent. For example, the layers including the first active agent and the second active agent may alternate with each other or may alternate with layers that do not include an active agent. The thin film may be disposed on a substrate having a texture having a size scale between about 100 nm and about 500 nm. The substrate may be a medal, ceramic, polymer, or semiconductor material. The thin film may include a buffer comprising a plurality of polyelectrolyte bilayers that are stable with respect to an applied voltage and disposed between the plurality of alternating layers and a substrate. The layers of the thin film may delaminate sequentially in response to the second predetermined voltage. At least a portion of the film may be organized in tetralayer heterostructures including first and second layers having a first charge and the same composition and third and fourth layers interspersed with the first and second layers and having a second charge, wherein the third layer includes an active agent having a predetermined physiological target and the fourth layer includes a material that is inactive with respect to the predetermined target.

The film may be about 1 to 10 nm thick, between 10 and 100 nm thick, between 100 to 1,000 nm, thick, between 1,000 and 5,000 nm thick, or between 5,000 and 10,000 nm thick.

In another aspect, the invention is a drug delivery device including a support and a decomposable thin film disposed on the support. The drug delivery device may further include a first electrode and second electrode disposed on opposing sides of the decomposable thin film. The electrodes may be an electrical communication with a microprocessor that controls when a voltage is applied across the electrodes.

In another aspect, the invention is a method of generating a three-dimensional structure on a surface. The method includes providing a charged region on the surface, and assembling a plurality of layers of alternating charge on the surface. At least a portion of the layers exhibit a change in net charge upon a change in an applied voltage.

Assembling a plurality of layers may include immersing at least a portion of the surface in alternating solutions containing layer-forming materials of opposite charge, assembling a plurality of discrete pluralities of layers on the surface, or both. The discrete pluralities of layers need not all have the same composition. Assembling may include one or more of spray coating, ink-jet printing, brush coating, roll coating, spin coating, soft lithography, microcontact printing, multilayer transfer printing, layer-by-layer deposition, and roll-to-roll coating.

In another aspect, the invention is a method of controllably releasing a material from a thin film including a plurality of layers of alternating charge in which the material is disposed. The method includes changing an applied voltage from a first value to a second value at a predetermined frequency, wherein at least a portion of the layers exhibit a reduced net charge at the second value. Either the first value or the second value may be 0 V.

In another aspect, the invention is a method of controllably releasing material from a plurality of discrete thin films disposed on a surface, each thin film including a plurality of layers of alternating charge. The method includes applying a first predetermined voltage at a first predetermined frequency to a first predetermined member of the plurality of thin films, wherein at least a portion of the layers in the first predetermined member exhibit a reduced net charge at the first predetermined voltage.

The method may further include applying a second predetermined voltage at a second predetermined frequency to a second predetermined member of the plurality of thin films at a predetermined time interval following applying the first predetermined voltage, wherein at least a portion of the layers in the second predetermined member exhibit a reduced net charge at the second predetermined voltage. The first predetermined voltage and the second predetermined voltage may be applied to both the first predetermined member and the second predetermined member.

### BRIEF DESCRIPTION OF THE DRAWING

The invention is described with reference to the several figures of the drawing, in which,

FIG. 1 is a schematic illustrating an exemplary method of alternating layer-by-layer assembly.

FIG. 2 is a photograph illustrating the extended potential range of electrochromism of a 50 layer pair LPEI/PB film immersed in an electrochemical cell after 30 s equilibration at the indicated potential.

FIGS. 3 and 3A are schematics illustrating a chip incorporating an exemplary alternating layer-by-layer assembly.

FIG. 4 is a schematic illustrating an exemplary reaction for the production of Prussian Blue.

FIG. 5 is a series of transmission electron micrographs of Prussian Blue nanoparticles.

FIG. 6 is a graph showing the correlation of total film thickness to layer pair number for LPEI/PB films. The inset shows the correlation of roughness to layer pair number.

FIGS. 7A and B are graphs showing the current density of A) 10 layer pairs of LPEI/PB and B) 30 layer pairs during cyclic voltammetry, in which the arrow indicates increasing scan rate, which doubles (e.g., 25, 50, 100, 200, 400 mV/s) for each sequential curve.

FIGS. 7C and D are graphs showing the Faradiac charge response of A) 10 layer pairs of LPEI/PB and B) 30 layer pairs to C) oxidation from −0.2V to 0.6 V and D) reduction from 0.6 V to −0.2 V during square wave switching.

FIG. 8A is a graph showing the increase in absorbance of a LPEI/PB film at 700 nm as the applied potential is increased stepwise from −0.2V to 0.6V in 0.1 V steps. The inset shows absorbance at 700 nm and 0.6 V as a function of layer pair number.

FIG. 8B is a graph illustrating the absorbance of a LPEI/PB film as measured dynamically during switching between −0.2 V and 0.6 V, with 30 s at each potential.

FIG. 9A is a graph illustrating the current density of a 50 layer pair LPEI-PB film during cyclic voltammetry between −0.2 V and 1.5 V.

FIG. 9B is a graph illustrating the absorbance at 700 nm of a 50 layer pair LPEI/PB film during stepwise oxidation from 0.6 V to 1.5 V in 0.1 V steps.

FIG. 10A is a graph illustrating the absorbance at 700 nm of a 50 layer pair LPEI/PB film immersed in a quiescent cuvette well during switching between 0.6 V and 1.5 V.

FIG. 10B is a graph illustrating film thickness of a 50 layer pair LPEI/PB film after 25 minutes soaking in a shear cell under various conditions.

FIGS. 11A and B is a graph illustrating the thickness of a 15 tetralayer film of LPEI/Heparin/LPEI/PB versus the number of scans between 0.2 V and 1.2 V at the indicated scan rate.

### DETAILED DESCRIPTION OF CERTAIN PREFERRED EMBODIMENTS

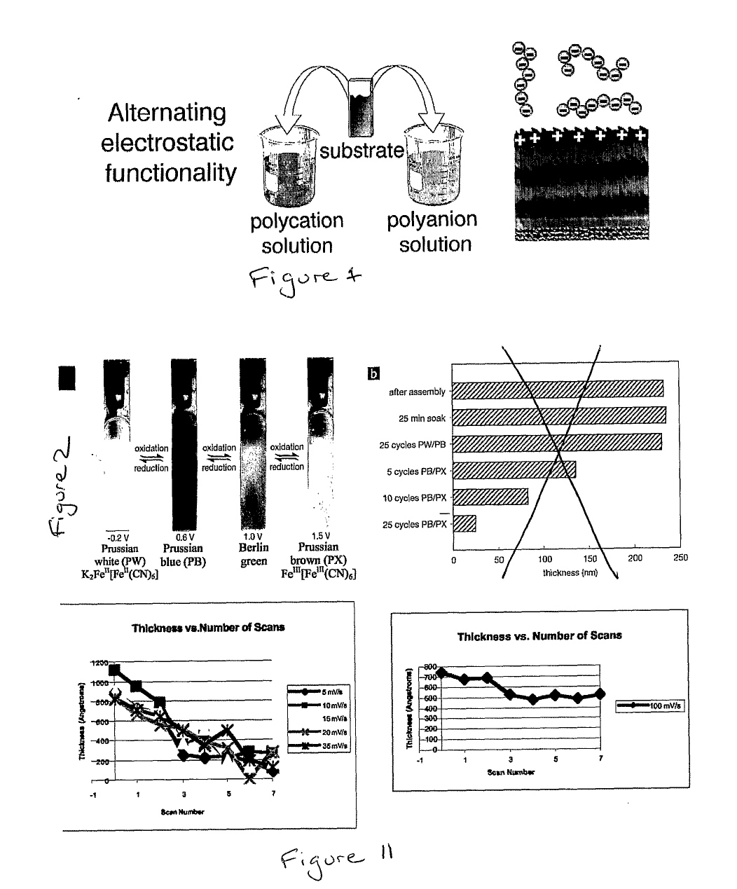

Layer-by-layer assembly (LBL), also known as polyelectrolyte multilayer assembly, is an approach based on the alternating adsorption of materials containing complementary charged or functional groups to form integrated ultrathin films [12-15], as illustrated schematically in FIG. 1. This method, which is most often manifest in the alternation of oppositely charged species, can be used to create highly tuned, functional thin films with nanometer level control of film composition and structure. Recently, there has been a virtual explosion in the number of functional polymers, nanoparticles, and organic and inorganic systems put into these films. Furthermore, it has been demonstrated that multilayer thin films can be produced in a variety of novel and unique geometries and forms using templating and patterning techniques that make these systems even more applicable to the creation of unique nanoassemblies [16,17]. We have exploited redox active nanoscale systems as systematically deconstructible multilayers for use as an electrochemically-controlled means of drug delivery.

In one embodiment, the invention is a decomposable thin film comprising a plurality of alternating positive and negative layers. The alternating layers are stable at a first applied voltage and decompose upon application of a second applied voltage. The thin film may have either the positive or negative layers, or a portion of either of these, fabricated from a polyelectrolyte, while the oppositely charged layers are a non-polymeric material. Alternatively, both the positive and negative layers may be polyelectrolytes. At least a portion of the positive or negative layers include a redox material that is rendered neutral by the application of a first voltage and brought back to a charged state by the application of a second voltage.

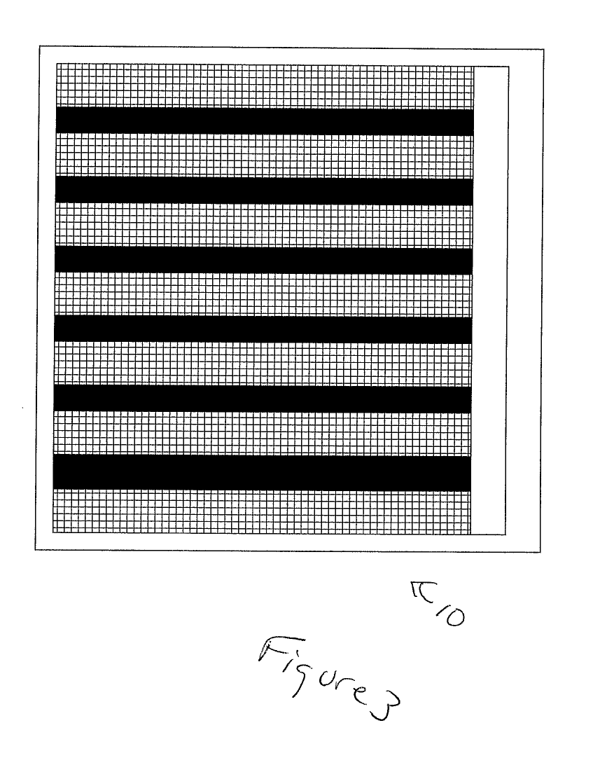

An exemplary redox material is Prussian Blue (PB). While Prussian Blue is not the only redox material suitable for use with the invention, it provides an excellent example for describing a possible mechanism of decomposition of the films. Upon application of a small electrochemical potential, the PB crystal proceeds through a series of increasing oxidation states: Prussian White (PW, K₂Fe^{II}[Fe^{II}(CN)₆]), Prussian Blue (KFe^{III}[Fe^{II}(CN)₆]), Prussian Brown (PX, also called Prussian Yellow, Fe^{III}[Fe^{III}(CN)₆]), and mixed PB and PX in an 1:2 ratio, called Prussian Green or Berlin Green (BG). As used herein, any thin film incorporating any of these materials will be said to include PB, but the PB within the films can be electrochemically switched to the PW, PB, PX, or BG states (FIG. 2). All of these are chemically and biologically stable, and PB is non-toxic and has long been known as one of the most effective treatments for thallium poisoning and radioactive metal contamination (Pearce, “Food Chem. Toxicol.” (1994), 32, 577).

In one embodiment, a thin film is built from alternating layers of negatively charged PB and a positively charged polymer such as polyethyleneimine (PEI). The film may be immersed in an aqueous solution and exposed to a voltage of about 1 V. This voltage oxidizes PB to PX, which has no surface or interior ionization in aqueous environments. As a result, the thin film is essentially composed of alternating positive and neutral layers depending on the pH (PEI is charged at a pH of about 4). Without wishing to be bound by any particular theory, it is thought that the PX particles are non-dispersable and hydrophobic and do not quickly diffuse from the film surface into the aqueous electrolyte environment. As a result, it is thought that film dissolution proceeds because the PX particles cannot provide charge compensation for the PEI, which desorbs because of charge-charge repulsion within and possibly between PEI layers. The desorbing PEI chains may carry off the PX particles. If the voltage is decreased, the PX is reduced to PB and the film stops degrading.

In one embodiment, then, the thin film comprises alternating positive and negative layers, at least one of which includes a material that has a neutral redox state. In general, anionic polyelectrolytes may be polymers with anionic groups distributed along the polymer backbone. The anionic groups, which may include carboxylate, sulfonate, sulphate, phosphate, nitrate, or other negatively charged ionizable groups, may be disposed in groups pendant from the backbone, may be attached to the backbone directly, or may be incorporated in the backbone itself. The cationic polyelectrolytes may be polymers with cationic groups distributed along the polymer backbone. The cationic groups, which may include protonated amine, quaternary ammonium or phosphonium derived functions or other positively charged ionizable groups, may be disposed in groups pendant from the backbone, may be attached to the backbone directly, or may be incorporated in the backbone itself.

For example, a range of hydrolytically degradable amine-containing polyesters bearing cationic side chains have recently been developed (Putnam et al. Macromolecules 32:3658-3662, 1999; Barrera et al. J. Am. Chem. Soc. 115:11010-11011, 1993; Kwon et al. Macromolecules 22:3250-3255, 1989; Lim et al. J. Am. Chem. Soc. 121:5633-5639, 1999; Zhou et al. Macromolecules 23:3399-3406, 1990; each of which is incorporated herein by reference). Examples of these polyesters include poly(L-lactide-co-L-lysine) (Barrera et al. J. Am. Chem. Soc. 115:11010-11011, 1993; incorporated herein by reference), poly(serine ester) (Zhou et al. Macromolecules 23:3399-3406, 1990; which is incorporated herein by reference), poly(4-hydroxy-L-proline ester) (Putnam et al. Macromolecules 32:3658-3662, 1999.; Lim et al. J. Am. Chem. Soc. 121:5633-5639, 1999; each of which is incorporated herein by reference), and more recently, poly[α-(4-aminobutyl)-L-glycolic acid]. Additional exemplary positively charged polyelectrolytes include both linear and branched PEI (LPEI and BPEI), polyallylamine HCI (PAH), polylysine, chitosan, poly(diallydimethylammonium chloride) (PDAC), polysaccharides, polymers of positively charged amino acids, polyaminoserinate, hyaluronan, and poly beta amino esters such as those disclosed in U.S. Ser. No. 09/969,431, filed Oct. 2, 2001, entitled “Biodegradable poly(P-amino esters) and uses thereof” and Lynn et al., J. Am. Chem. Soc. 122:10761-10768, 2000, the entire contents of both of which are incorporated herein by reference.

Exemplary polyanions include polymalic acid, hyaluronic acid, polymers of negatively charged and acidic amino acids and polynucleotides. The polyelectrolytes need not be biodegradable.

Conducting polymers and redox polymers may be used as a redox material or as polyelectrolytes. Exemplary polymers include but are not limited to polypyrrole, polyaniline, polythiophene, polyporphyrins, poly(siloxane), PEI, poly(ethylene oxide), poly(vinyl pyridine), polyheme, and polymers including phthalocyanines, metal complexes of cyclams and crown ethers, and pyridyl, bipyridyl, and polypyridyl complexes of transition metals. In addition, dendrimers may be used as a redox material or as a cationic or anionic polymer. Exemplary dendrimers may be formed from polyesters, poly(propylene imine), porphyrins, polylysine, poly(ethylene oxide), polyethers, poly(propyl amine), and other materials known to those skilled in the art. Dendrimers may be fabricated to have polar or positively or negatively charged surface groups such as carboxylate, hydroxyl, and amine, as described in PCT Publications Nos. WO95/34595 and WO98/03573, the contents of which are incorporated herein by reference, or may be complexed to have various active groups at their surfaces, as described in U.S. Pat. No. 5,714,166, the contents of which are incorporated herein by reference. Additional dendrimers include the poly(amido amine) (PAMAM) dendrimers, which are available with a variety of cores and surface groups from Sigma-Aldrich.

The thin films for use with the invention may be built up to any desired thickness simply by adding additional layers. In one embodiment, the films are between 1 and 10,000 nm thick, for example, between 1 and 10 nm, between 10 and 100 nm, between 100 and 1000 nm, between 1000 and 5000 nm, or between 5000 and 10,000 nm thick.

The thin films for use with the invention may be used to deliver a variety of biologically active agents. If these materials are charged, they may simply be incorporated as layers within the film. For example, nucleosides and polynucleotides are intrinsically charged and may be directly incorporated into a film. Particulate gene delivery systems are often positively charged at physiological pH. For example, cationic liposomes may be used to encapsulate DNA or RNA. If the material being delivered is not charged, it may be encapsulated for incorporation into the film. For example, nanoparticles of PLGA or other charged polymers may be used to encapsulate various materials (see Freiberg, S., et al., Int. J Pharm. (2004) 282: 1-18). Various techniques for modifying the surface chemistries of PLGA and similar microparticles are known to those skilled in the art and may be used to modify the surface charge of particles for incorporation into the film (see Pfeifer, et al., Biomaterials, (2005) 26:117-124). Micelles or dendrimers may also be used to encapsulate uncharged biologically active materials, e.g., hydrophobic small molecules and some proteins and growth factors. Micelles may be constructed of materials that can present an accommodating, e.g., hydrophobic, environment to the material and a charged outer surface that allows the micelle to be incorporated into a layer. Both linear and spherical dendrimers with hydrophobic and charged regions are also available. Techniques for encapsulating various materials with dendrimers are well known to those skilled in the art. Even charged materials may be encapsulated to increase their stability or to change the layer into which they are incorporated. For example, many drugs, such as heparin and chondroitin sulfate, are negatively charged. In some embodiments, globular proteins having a net charge may be incorporated into films. One skilled in the art will recognize that the pH and ionic strength of the film may be coordinated with the composition of the proteins so the proteins will configure themselves to expose the appropriately charged amino acids for the particular layer they are disposed in.

The biologically active agents to be incorporated in the thin films according to the invention may be therapeutic, diagnostic, prophylactic or prognostic agents. Any chemical compound to be administered to an individual may be delivered. The active agent may be a small molecule, organometallic compound, nucleic acid, protein, peptide, metal, an isotopically labeled chemical compound, drug, vaccine, immunological agent, etc. Exemplary biologically active agents include small molecules, biomolecules, and bioactive agents as defined herein.

In one embodiment, the biologically active agents agents are organic compounds with pharmaceutical activity. In another embodiment of the invention, the agent is a small molecule that is a clinically used drug. In exemplary embodiments, the drug is an antibiotic, anti-viral agent, anesthetic, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, adrenergic blocking agent, diuretic, cardiovascular active agent, vasoactive agent, non-steroidal anti-inflammatory agent, nutritional agent, etc.

In another embodiment, the biologically active agent is a protein drug, such as an antibody, an antibody fragment, a recombinant antibody, a recombinant protein, a purified protein, a peptide, an amino acid and combinations thereof. Exemplary protein drugs include but are not limited to biologically active macromolecules such as enzyme inhibitors, colony-stimulating factors, plasminogen activators, polypeptide hormones, insulin, myelin basic protein, collagen S antigen, calcitonin, angiotensin, vasopressin, desmopressin, LH-RH (luteinizing hormone-releasing hormone), somatostatin, glucagon, somatomedin, oxytocin, gastrin, secretin, h-ANP (human atrial natriuretic polypeptide), ACTH (adrenocorticotropic hormone), MSH (melanocyte stimulating hormone), beta-endorphin, muramyl dipeptide, enkephalin, neurotensin, bombesin, VIP (vasoactive intestinal peptide), CCK-8 (cholecystokinin), PTH (parathyroid hormone), CGRP (calcitonin gene related peptide), endothelin, TRH (thyroid releasing hormone), interferons, cytokines, streptokinase, urokinase, and growth factors. Exemplary growth factors include but are not limited to activin A (ACT), retinoic acid (RA), epidermal growth factor, brain-derived neurotrophic factor, keratinocyte growth factor, cartilage growth factors, bone morphogenetic protein, platelet derived growth factor, hepatocyte growth factor, insulin-like growth factors (IGF) I and II, hematopoietic growth factors, peptide growth factors, erythropoietin, angiogenic factors, anti-angiogenic factors, interleukins, tumor necrosis factors, interferons, colony stimulating factors, t-PA (tissue plasminogen activator), G-CSF (granulocyte colony stimulating factor), heparin binding growth factor (HBGF), alpha or beta transforming growth factor (α- or β-TGF), fibroblastic growth factors, epidermal growth factor (EGF), vascular endothelium growth factor (VEGF), nerve growth factor (NGF) and muscle morphogenic factor (MMP). Also suitable for use with the invention are recombinantly-produced derivatives of therapeutically useful proteins, including deletion, insertion and substitution variants, which on the whole have similar or comparable pharmacological properties.

In one embodiment, the biologically active agent delivered using the techniques of the invention is a nucleic acid based drug, such as DNA, RNA, modified DNA, modified RNA, antisense oligonucleotides, expression plasmid systems, nucleotides, modified nucleotides, nucleosides, modified nucleosides, nucleic acid ligands (e.g. aptamers), intact genes, a promoter complementary region, a repressor complementary region, an enhancer complementary region, and combinations thereof. A promoter complementary region, a repressor complementary region, or an enhancer complementary region can be fully complementary or partially complementary to the DNA promoter region, repressor region, an enhancer region of a gene for which it is desirable to modulate expression. For example, it may be at least 50% complementary, at least 60% complementary, at least 70% complementary, at least 80% complementary, at least 90% complementary, or at least 95% complementary.

The thin films may be produced using layer-by-layer deposition techniques. In one embodiment, the thin films are produced by a series of dip coating steps in which a substrate is dipped in alternating solutions containing the components of the cationic and anionic layers (FIG. 1). The thickness of the films may be controlled by adjusting the pH or ionic strength of the dipping solution, as described in Shiratori, et al., Macromolecules (2000) 33: 4213-4219, the entire contents of which are incorporated herein by reference, so that the thickness of the various layers may be varied between 0.5 nm and 80 nm as desired. Indeed, multiple dipping solutions may be employed, so that the thickness of the layers may be varied across the film. Additionally or alternatively, it will be appreciated that deposition may be performed by spray coating, inkjet printing, brush coating, roll coating, spin coating, soft lithography, microcontact printing, multilayer transfer printing, polymer-on-polymer printing or combinations of these techniques. A variety of techniques are described in Hammond, Adv. Mater. (2004) 16: 1271, the entire contents of which are incorporated herein by reference. For example, a roll-to-roll coating method recently developed by Avery Dennison can produce 30 bilayer pair films at 60 feet per second.

The thin films may be deposited on practically any substrate. A variety of materials can be used as substrates of the present invention such as, but not limited to, metals, e.g., gold, silver, platinum, and aluminum; metal-coated materials; metal oxides; plastics; ceramics; silicon; glasses; mica; graphite; hydrogels; polymers such as polyamides, polyphosphazenes, polypropylfumarates, polyethers, polyacetals, polycyanoacrylates, polyurethanes, polycarbonates, polyanhydrides, polyorthoesters, polyhydroxyacids, polyacrylates, ethylene vinyl acetate polymers and other cellulose acetates, polystyrenes, poly(vinyl chloride), poly(vinyl fluoride), poly(vinyl imidazole), poly(vinyl alcohol), poly(ethylene terephthalate), polyesters, polyureas, polypropylene, polymethacrylate, polyethylene, poly(ethylene oxide)s and chlorosulphonated polyolefins; and combinations thereof. For example, a substrate of one material may be coated with a second material, or two materials may be combined to form a composite.

It will be appreciated that materials with an inherently charged surface are particularly attractive substrates for LBL assembly of an inventive thin film. Alternatively, a range of methods are known in the art that can be used to charge the surface of a material, including but not limited to plasma processing, corona processing, flame processing, and chemical processing, e.g., etching, micro-contact printing, and chemical modification. For example, plastics can be used as substrates, particularly if they have been chemically modified to present polar or charged functional groups on the surface. Additionally or alternatively, substrates can be primed with specific polyelectrolyte bilayers such as, but not limited to, LPEI/SPS, PDAC/SPS, PAH/SPS, LPEI/PAA, PDAC/PAA, and PAH/PAA bilayers (SPS=poly(styrene sulfonate), PDAC=poly(diallyldimethyl ammonium chloride), PAH=poly(allylamine hydrochloride), PAA=poly(acrylic acid), LPEI=linear poly(ethylene imine)) that form readily on weakly charged surfaces and occasionally on neutral surfaces. It will be appreciated that primer layers provide a uniform surface layer for further LBL assembly and are therefore particularly well suited to applications that require the deposition of a uniform thin film on a substrate that includes a range of materials on its surface, e.g., an implant or a complex tissue engineering construct.

One of the advantages of the techniques of the invention is that a thin film may be formed on any shape or texture substrate. Exemplary shapes that the substrate may take include particles, tube, sphere, strand, coiled strand, and capillary network, sponge, cone, portion of cone, rod, strand, coiled strand, capillary network, film, fiber, mesh, sheet, or threaded cylinder. In addition, the films may be formed on textured substrates. Substrates may have inherent roughness or may be roughened using techniques such as sanding, filing, plasma etching, chemical etching, dewetting, and mechanical pitting (for example, by sandblasting). Alternatively or in addition, substrates may have machined or natural macrotexture such as bumps, grooves, raised ridges, teeth, threads, wedges, cylinders, pyramids, blocks, dimples, holes, or grids. The texture may have a lateral resolution of about 500 nm to about 100 nm or smaller.

The composition of the thin film may be varied through the thickness of the film The composition may be varied to control the degradation rate, the rate of release of a particular agent, or both. For example, the amount of an active agent in the various layers may be adjusted so that more of the agent is released by the upper layers of the film than the inner layers, or vice versa. Cyclical release profiles may also be created by separating drug-containing layers with blocks of passive polymer layers.

Alternatively or in addition, the thin film may be constructed to release more than one active agent. The agents may be released sequentially, or one agent may be phased in as the dosage of the other is decreased. Where it is desired to administer two or more agents sequentially, it may be desirable to have several “blank” layers, for example between 4 and 40 layer pairs, in between blocks of layers containing consecutive agents. Integrated films may also be constructed in which a gradient delivery is achieved between the introduction of a first and second drug. For example, two drugs may be directly alternated in a heterostructure, which varying degrees of “overlap” between the first and second drug.

Once the thin film is assembled and the active agent incorporated therein, the coated substrate may be implanted in a tissue site. The active agent is released by exposing the film to an electric field. In one embodiment, the film may be connected to leads that allow a voltage to be applied across the film. In one configuration, the active agent is incorporated into microchips as described in U.S. Pat. No. 5,797,898. FIG. 3 shows an exemplary microchip for use with an embodiment of the invention. FIG. 3A shows an exploded view of microchip 10, including substrate 11, electrode 12 having solid projections 14, film 16, and electrode 18 having gridded projections 20. Gridded projections 20 extend across the microchip, so that the material underneath the grid can eventually leave the microchip. The projections 14 and 20 are oriented perpendicular to one another, although they lie in two plains, and the loci where they cross define areas where an electric field can be applied across a cross-section of the film. The spaces in and between the gridded electrodes may be filled with an anode material that oxidizes and dissolves upon application of the field. The anode material can increase the lifetime of the chip by preventing materials from diffusing into or out of the film before an electric field is applied to cause delamination of the thin film. One skilled in the art will recognize that the electrodes may be set up with different configurations. For example, switches may be employed to apply a voltage across only selected areas of the film.

The thin film may be cast on the chip as a unit, or masking techniques may be used to create individual film islands. The material that is not deposited in a well may be easily removed from the surface after the electrodes are deposited by rinsing the surface with an acidic or basic solution in which either the positively or negatively charged layers become neutrally charged.

In one embodiment, a plurality of thin films are deposited on a substrate that has been prepared to deliver a voltage to an array of individual sections of the film. In such an embodiment, it may be desirable to deposit the thin films using soft lithography techniques or other techniques that do not require that the entire surface be coated with the same material. For example, a stamp, e.g., of poly(dimethyl siloxane), having raised portions in the pattern in which the thin films or a portion of the thin films are to be deposited may be used to transfer material from reservoirs to the substrate. A series of reservoirs may be prepared containing solutions of, e.g., polyelectrolytes, redox agents, and/or drugs. For example, a circuit may be prepared on the substrate, following which contact printing techniques such as those described in U.S. Pat. Nos. 5,512,131 and 6,180,239, the contents of both of which are incorporated herein by reference, may be used to deposit an array of thin film “stacks” on a circuit. Indeed, the circuit itself may be prepared using soft lithography techniques. Methods of depositing a circuit on the surface are also described in U.S. Pat. No. 6,123,681, the contents of which are incorporated herein by reference. By depositing multiple thin films on a substrate together with circuitry to individually control each film, a complicated regimen of active agents may be administered at doses that may vary over time. Indeed, the substrate may also include a microprocessor and a power supply to control the degradation of the films and the administration of the various active agents. Alternatively, the substrate may include a receiver, and a radio or other frequency signal may be administered externally from a patient to control the voltage administered to the individual thin films.

It is not necessary to dissolve an entire film all at once. The film will degrade only so long as the appropriate voltage is applied to render the redox material neutral. The degradation of the film and the release of an active agent from the film may be pulsed by cycling the applied voltage to alternate between the neutral and charged states of the redox material. Indeed, these techniques are especially useful where it is most convenient to implant a thin film during a particular surgical procedure, but it is not necessary to deliver the active agent for some period of time. The agent will not diffuse from the film, and the delivery of the agent may be turned on at an arbitrary time after the surgery, obviating a second procedure to deliver a drug releasing material.

### EXAMPLES

### Example 1

### Production of Prussian Blue

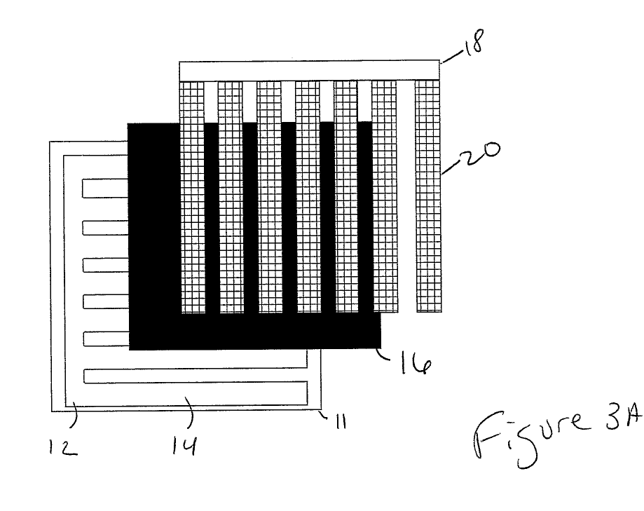

Based on analogy to polymer step-growth mechanisms, stoichiometric parity between reactants is a condition for large, uniform crystal growth (though the analogy is not exact because PB synthesis is a heterogeneous phase polymerization). If the reactant ratio is adjusted to include a large excess of a single reactant, it is possible to reduce crystal size. Our recipe employed a 5:1 molar ratio of potassium ferricyanide to iron(II) chloride. The simple reaction is shown in FIG. 4, with intercalated potassium ions within the crystal omitted for clarity. PB was synthesized by the addition of 35 mL of a 0.01 M aqueous solution of FeCl₂ (Aldrich) dropwise to a 35 mL solution containing 0.05 M potassium ferricyanide (Aldrich) and 0.05 M KCl. After complete addition, the liquid was vigorously agitated for 1 min and then immediately subjected to ultrafiltration against a 3000 Da regenerated cellulose membrane to remove the large excess of potassium ferricyanide. Filtration was performed using an Amicon 50 mL volume ultrafiltration apparatus with magnetic agitator. A Millipore membrane with nominal molecular weight cutoff of 3 k and constructed of regenerated cellulose was used in the filtration. Filtrate side hydrostatic pressure was maintained at 50 psi for approximately 48 h for the elution of at least 1000 mL of permeate (10 equivalent volumes). Permeate color was initially yellow, turning to clear after 250 mL of permeate was collected indicating the removal of excess potassium ferricyanide. Absence of any blue hue in the permeate confirmed that no significant amount of PB passed the membrane. The ultrafiltration retentate was used in LBL assembly as collected immediately after filtration, after pH adjustment to pH 4 by the addition of several drops of a saturated aqueous solution of potassium hydrogen phthalate.

This synthesis and processing scheme produced dark blue aqueous suspensions that did not scatter light and were stable indefinitely without agitation or stabilizer addition. The long-term stability of these dispersions indicated that the PB particles were primarily KFe^{III}[Fe^{II}(CN)₆] and were therefore ionization-stabilized. Particle size was determined using transmission electron microscopy (TEM), as shown in FIG. 5. The lower magnification image FIG. 5A shows a dried droplet of PB suspension in which several particle sizes are apparent. Larger crystals of approximately 6-10 nm could be magnified; the FCC crystal lattice of PB is clearly visible in FIG. 5B, C. In FIG. 5C, the square aspect that is common to transition metal hexacyanoferrate crystals is clearly depicted. From this evidence and the observation of high stability we therefore identify the dispersion as consisting of polydisperse PB particles and crystals of the “soluble” type with size ranging from 15 nm to <1 nm, and a median size of 4-5 nm.

### Example 2

### Production of PB/LPEI Multi Layers

The anionically ionized PB suspension was LBL assembled with the weak (pH-sensitive) polycation linear poly(ethylene imine) (LPEI) at pH 4. We have previously shown that LPEI can increase ionic conductivity by two orders of magnitude over LBL films assembled using other weak polycations in films used as the solid or gel electrolyte layer in electrochemical power storage devices.^{[18]} LPEI was therefore chosen for assembly with PB to capture this benefit for acceleration of electrochromic switching speed.

Aqueous solutions containing dissolved polycation LPEI (Poly-sciences MW 25 k) were formulated at 10 mM with respect to the poly-electrolyte equivalent weight (weight of ionized repeat unit). The pH was adjusted to pH4 using sodium hydroxide and hydrochloric acid solutions. ITO-glass substrates with dimensions 0.7 cm×5 cm (Delta Technologies, 6 Ω/square) were cleaned by ultrasonication in a series of solvents: dichloromethane, methanol, acetone, and Milli-Q water for 15 min each, followed by a 5 min oxygen plasma etch (Harrick PCD 32G) to provide a clean, hydroxyl-rich surface.

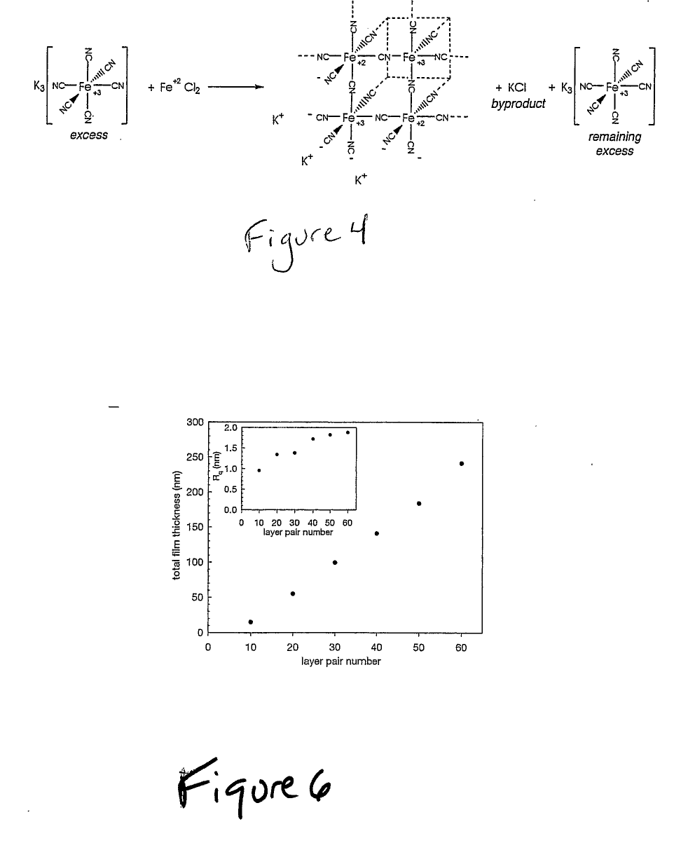

The assembly of LPEI/PB nanocomposites proceeded with the exposure of clean indium tin oxide (ITO)-coated glass to 1) an aqueous LPEI solution for 10 min; 2) a 4 min water rinse; 3) the aqueous PB dispersion for 10 min; and 4) another 4 min water rinse. Film assembly was automated with a Carl Zeiss HMS DS-50 slide stainer. This four-step exposure sequence results in the deposition of a single layer pair, and was repeated for the nominal required number of layer pairs using a robotic system. Film growth atop the ITO substrate was apparent with the visual observation of smooth, pale blue films after 10 layer pairs had been deposited. Thickness measurements on ITO substrates were performed with a Tencor P10 profilometer by scoring the film and profiling the score. A tip force of 5 mg was used to avoid penetrating the polymer film. The thickness increase profile of the LPEI/PB system is shown in FIG. 6. Thickness increases in a reproducibly linear fashion at approximately 4.1 nm per layer pair, and RMS (root mean square) roughness (as evaluated by profilometry) remains less than 2 nm even up to 60 layer pairs. Atomic force microscopy (AFM) images (not shown) of a 60 layer pair LPEI/PB film indicated a RMS roughness of 4.5 nm. This value is larger and more accurate than that indicated by profilometry because the smaller AFM probe tip could sample more surface features.

Given the layer pair dimensions, the size of depositing PB particles should be no greater than 4 nm in diameter, a value consistent with the deposition of PB as a layer of single crystals.

### Example 3

### Electrochemical Analysis of PB/LPEI Multilayers

Once assembled, the LPEI/PB series was subjected to electrochemical analysis. Electrochemical analysis was performed using an EG&G 263 A potentiostat/galvanostat. These measurements were performed in a flat cell of 30 mL volume and approximately 0.3 cm² working electrode area. The electrolyte used was aqueous 0.1 M potassium hydrogen phthalate with a pH of exactly 4. The counterelectrode was 4 cm² platinum foil, and reference was a K-type saturated calomel electrode.

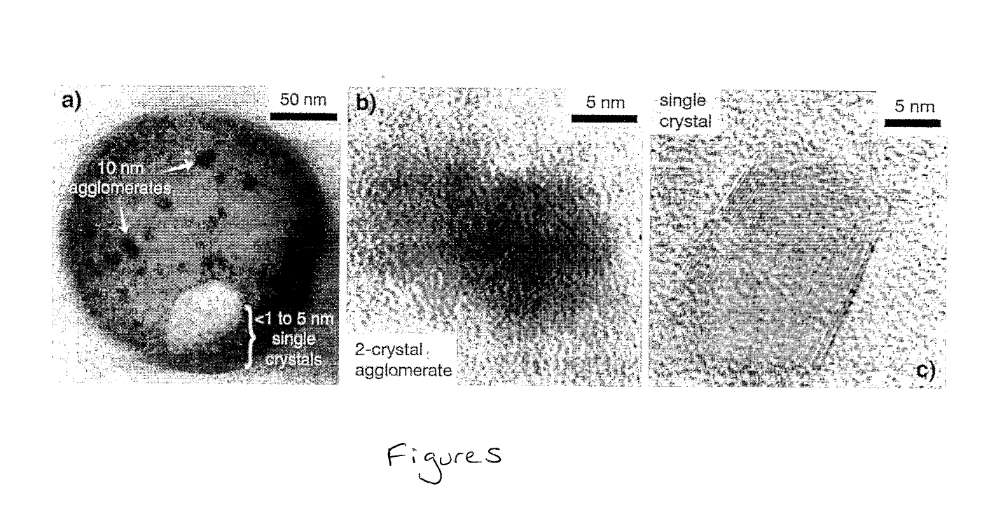

Cyclic voltammetry (CV) was performed around the potential range expected for the PW⇄PB transition: between −0.2 and 0.6 V at scan rates of 25, 50, 100, 200, and 400 mV s⁻¹. Some representative CVs are shown in FIGS. 7A,B. They exhibit the reversible PW⇄PB transition at an E_{1/2} of approximately 0.15 V, a value consistent with PB electrochemistry described elsewhere, although slightly more cathodic than the 0.2 V that are typically reported.^{[19-21]} The redox potential may be shifted because LPEI has replaced potassium as the counterion for the particle surface and accessible interior. In the case of LPEI/PB, reduction of PB to PW must be accompanied by potassium ion insertion from the electrolyte (0.1 M potassium hydrogen phthalate) because LPEI cannot supply sufficient cationic ionization to compensate for the doubling of PB particle anionic ionization that occurs with reduction. This potassium ion insertion may be less thermodynamically favored when the particle surface is covered with LPEI, leading to greater stability of the more oxidized PB state. The substitution of polyvalent metal countercations for potassium has been shown to influence PB redox potential in a similar manner.^{[33]}

The redox peaks shown in FIG. 7A, B are broader than those observed for completely inorganic PB films.^{[19,20]} This general peak-broadening effect in electrochemically active films has been attributed by Peerce and Bard to the existence of non-equivalent electrochemical sites.^{[22]} It follows that LPEI/PB may possess a more distributed site character than inorganic PB because the environment of each redox site differs depending on the extent of its interactions with LPEI. Just as for other LBL-assembled electrochromic films,^{[23]} increasing skew in the LPEI/PB CVs is indicative of increasing resistance in the film due to increasing thickness, an effect that has also been observed in pure PB films.^{[19,20]} In general, peak height appears to increase linearly with scan rate, indicating that electrochemistry is not diffusion-controlled, though there is some deviation from this trend for the thickest films.

The second test applied to the LPEI/PB series was the application of a square wave switch between oxidizing (PB, 0.6 V) and reducing (PW, −0.2 V) potentials. The results of this square wave test are shown in FIG. 7C, D. The charge density increases up to a point and then asymptotically approaches an equilibrium value. Slower approach times for greater layer pair numbers can be observed. This final charge density may be taken as the full Faradaic charge density (relating to charge transferred directly into the film) because capacitative contributions and parasitic currents have been subtracted from these results. There exists a linear increase in the Faradaic charge density of the film as it increases linearly in thickness. The existence of a linear trend indicates that PB within the film remains completely electrochemically accessible even up to 60 layer pairs, implying that there exists some continuity to the interconnection of PB particles that allows charge percolation by sequential redox exchanges throughout the film thickness. The PB is not electrochemically switching only at the electrode surface, but rather a unified electrochemical response is observed from the entire film. This result was confirmed later in this work using spectroscopic measurements.

Based on the linear trend in Faradaic charge uptake of these LBL assembled films, it is possible to estimate the concentration of redox centers within the LBL structure. This concentration is 4.6 mmol cm⁻³ based on a linear fit of the Faradaic charge density to film thickness. The redox center concentration is higher than that of many electrochemically active polymers because PB density is greater at 1.75-1.80 g cm⁻³.^{[24]} Utilizing the concentration of redox centers, and employing the PB density, it is possible to determine a compositional profile for the LPEI/PB system because each redox center in the film is rigorously defined as a single PB unit crystal. A concentration of 4.6 mmol cm⁻³ PB within the film corresponds to a PB gravimetric loading of 1.2 g cm⁻³ PB, or a PB volume fraction of 0.68. Assuming that the density of LPEI remains 1.2 g cm⁻³, then the concentration of LPEI molar repeats in the film would be 6.1 mmol cm⁻³. This calculation leads to a LPEI/PB unit pairing ratio of 1.48, or approximately three LPEI repeats, (CH₂CH₂NH) or (CH₂CH₂NH₂)⁺, per every two PB unit crystals Fe^{III}[Fe^{II}(CN)₆]⁻. This ratio is consistent with electrostatic pairing of “soluble” PB and ionized LPEI because the LPEI repeats would be only partially ionized at the assembly pH of 4.^{[18]} If the particles were entirely “insoluble” PB, the LPEI/PB repeat unit molar ratio would be 1.41 rather than 1.48. This compositional range, which assumes only two density values and is therefore presumably quite reliable, does not indicate an absolute ratio of ion pairing because any PB in the “insoluble” form is non-ionic and the interior anionic sites of PB may not be accessible.

### Example 4

### Electrochromic Analysis of PB/LPEI Multilayers

The electrochromic color change of the LPEI/PB series was evaluated using spectroelectrochemistry, a tool based on the in situ collection of a UV-vis absorbance spectrum exhibited by an electrode film at various equilibrium potentials while it is immersed in a cuvette. Double potential step chronoamperometry was performed by stepping between −0.2 V and 0.6 V versus a standard calomel electrode (SCE), with 30 s per step and 60 s per cycle, with approximately 20 cycles performed sequentially before the measurement cycle. Spectroscopic characterization was performed with a StellarNet EPP2000 concave grating UV-vis-NIR spectrophotometer with combined incandescent and deuterium lamp sources. For spectroelectrochemistry, potential control was provided by EG&G 263 A, with the polymer-coated ITO-glass substrate positioned in a quartz cell and immersed in electrolyte, along with a platinum wire counter electrode, and SCE reference.

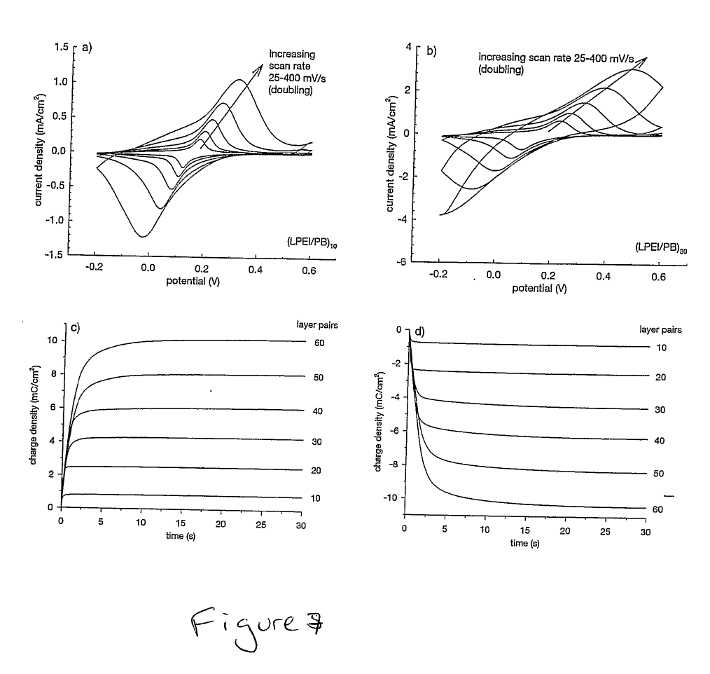

The spectroelectrochemistry of a 50 layer pair LPEI/PB film is shown in FIG. 8A. Beginning at −0.2 V, the composite is in the PW state, exhibiting almost no visible absorbance. As the electrode potential becomes more anodic, absorbance increases, with the largest increase occurring between 0.1 V and 0.2 V, a range that spans the formal redox potential of 0.15 V determined from CV. Fully oxidized, cyan-colored PB is attained at 0.3 V, and has a well-defined charge transfer band with an absorbance peak at 700 nm, which is consistent with that previously observed in fully inorganic PB films.^{[19,20,25]} The peak absorbance of oxidized PB shown in the FIG. 8A inset increases linearly with increasing layer pair number, indicating a consistent and continuous PB extinction coefficient and again, that all PB is redox-active within the film. Given the linearity in thickness, Faradaic charging, and absorbance for the LPEI/PB system, the molar extinction coefficient for a PB unit cell can be calculated as 8700 M⁻¹ cm⁻¹ at 700 nm.

The electrochromic performance of the LPEI/PB series was assessed with a focus on two key benchmarks: response time and contrast. The best performance relative to inorganic PB films was achieved at the high thicknesses that are most relevant to applications. Thick LPEI/PB nanocomposite films exhibit switching speeds competitive with inorganic PB while delivering superior contrast. Thus the LBL assembly of PB dispersions may be a superlative processing method to provide transition metal hexacyanoferrate films for application in electrochromic windows or displays.

Switching speed was evaluated using fast UV-Vis measurements to capture dynamic changes in absorbance with an applied potential square wave. Optical switching between the PW (low absorbance, −0.2 V) and PB (high absorbance, 0.6 V) is shown in FIG. 8B. Thinner films switch fast, with profiles resembling the applied potential square wave, while thicker films have more rounded and slower switching profiles. The evenly incrementing peak absorbance in FIG. 8A, B emphasizes the potential for achieving a desired film thickness, charge capacity, optical absorbance, and overall performance by essentially “dialing in” a layer pair number.

Switching duration is defined here as the time required to attain an absorbance shift that is 90% of the total absorbance span. Using this metric, switching time was calculated as reported in Table 1. For comparison, there exist several descriptions of inorganic PB response times; some report 100-500 ms^{[20,26,27]} while others report 5-10 s.^{[28-30]} This disparity is attributable to the thicknesses of the films tested. Faster switching films are thinner, with a Faradaic charge density of <4 mC cm⁻², while slower films are thicker. This comparison leads to two conclusions: 1) thinner LPEI/PB films respond slower than thin inorganic PB films, and 2) thicker LPEI/PB films respond faster than thick fully inorganic PB films. Here “thin” and “thick” more accurately describe a lesser or greater extent of planar Faradaic charge density of PB within the film (thickness is less accurate because of the density difference between LPEI/PB nanocomposite and inorganic PB). The relatively slower switching speed of thinner LPEI/PB films must be due to the electronically insulating nature of LPEI, which may hinder charge transfer from the ITO electrode to PB, a disadvantage compared to thin inorganic PB films that are in intimate contact with both electrode and electrolyte. The relatively faster switching speed of thicker LPEI/PB films is also attributable to the presence of the LPEI matrix, which should provide fast potassium ion migration to/from PB crystal surfaces within the film interior, a mechanism superior to the long-range potassium intercalation that would be expected of a thick monolithic PB film. We would expect accelerated switching from LPEI/PB with the usage of a more concentrated electrolyte; future studies will be performed at higher salt concentrations to evaluate advantages.

The contrast exhibited by LPEI/PB LBL assembled films is superior to those previously reported for inorganic PB films. Contrast is reported in Table 1 as the difference between the PW and PB state transmittance. An optimum thickness with maximum contrast is expected to lie beyond 60 layer pairs. In this study, the LPEI/PB contrast exceeds 77% and appears to approach 80% or greater. For comparison, previous descriptions of (presumably optimized) inorganic PB single films have reported contrasts of: 16%,^{[21]} 50%,^{[31]} and 57%,^{[26]} while electrochromic cells incorporating PB electrodes exhibit contrasts of 10%,^{[8]} 30%,^{[29,30]} and 60%,^{[32]} From comparison with previous reports, it appears that LPEI/PB contrast is superior because its bleached state is more transparent. Without being bound by any particular theory, we believe the increased transparency to be the result of two factors: 1) the smoothness of LPEI/PB films eliminates reflection and scatter, and 2) the small size of the PB particles and their dispersion within the LPEI matrix may allow for a greater bleaching extent because potassium ion intercalation distance is limited to the nanoparticle diameter.

TABLE 1

| Layer Pairs | Color time(s) | Bleach time(s) | Δ % T (λ = 700 nm) |
|---|---|---|---|
| 10 | 0.63 | 0.83 | 16.5% |
| 20 | 1.55 | 2.15 | 37.6% |
| 30 | 2.84 | 2.78 | 54.0% |
| 40 | 3.98 | 4.98 | 63.3% |
| 50 | 4.60 | 5.10 | 72.4% |
| 60 | 5.48 | 5.88 | 77.4% |

### Example 5

### Controlled Dissolution of PB/LPEI Multilayers

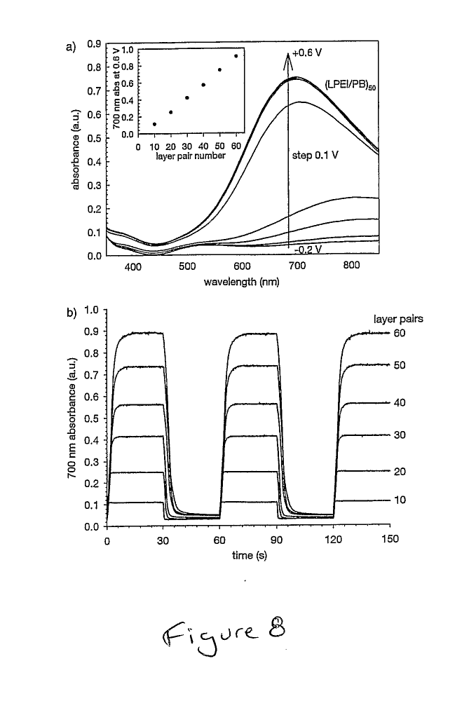

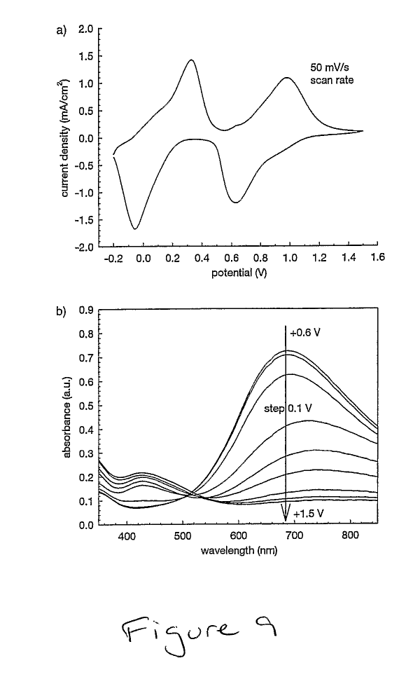

Given the encouraging electrochromic performance of LPEI/PB films for the PW⇄PB transition, the potential range of our study was expanded to more oxidative potentials to access additional states such as Fe^{III}[Fe^{III}(CN)₆] (PX). Initially, the feasibility of the transition was studied using cyclic voltammetry and spectroelectrochemistry. The CV waveform of a 50 layer pair film of LPEI/PB cycled from PW (−0.2 V) to PB (0.5 V) to PX (1.5 V) is shown in FIG. 9A. From this CV, the redox potential E_{1/2} of PB⇄PX is 0.85 V. Spectroelectrochemistry of the PB⇄PX transition is shown in FIG. 9B. The PB peak absorbance at 700 nm gradually decreases at more oxidizing potential, with a concomitant increase in absorbance at 450 nm that is the origin of the golden yellow PX coloration. The color state at ˜1.0 V, which features peaks at both 700 and 540 nm, is the intermediate BG state. The PX absorbance at 450 nm exhibits a molar extinction coefficient of 2550 M⁻¹ cm⁻¹, far lower than the 8700 M⁻¹ cm⁻¹ for PB state absorbance at 700 nm. It is for this reason that the PW⇄PB transition is most widely employed in electrochromic devices—PX presents far lower contrast. Photographs of LPEI/PB color states for a 50 layer pair film are shown in FIG. 2. The human eye can easily distinguish the many colors of this nanocomposite film.

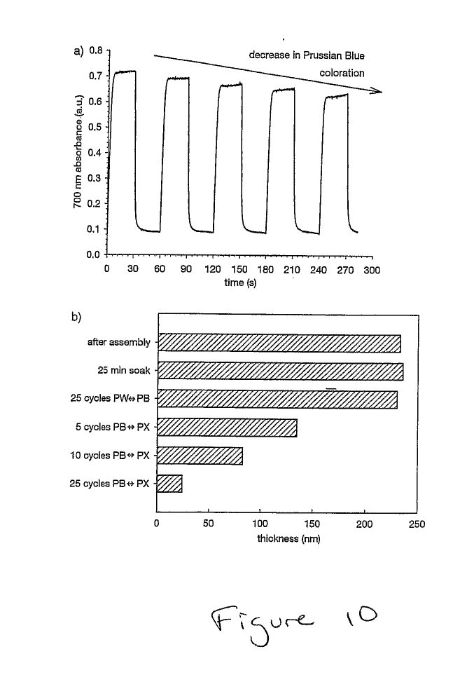

Although LPEI/PB promisingly exhibited multiple, differently colored absorbance states over an extended potential range, tests of optical switching revealed unexpected behavior. As can be seen in FIG. 10A, repeated switching of a 50 layer pair LPEI/PB film between 0.6 V (PB, high absorbance) and 1.5 V (PX, low absorbance) causes a steady decrease in overall absorbance with each cycle. This loss cannot be a result of chemical or electrochemical degradation—the PB, BG, and PX states are all chemically stable at these potentials, and control tests performed with other LPEI-based LBL assembled films such as LPEI/poly(acrylic acid) and LPEI/poly(styrene sulfonate) indicated no film degradation at these potentials. Clearly the film loses nanoparticles during switching. This particle loss must be explained by the electrochemical modulation of nanoparticle ionization during switching. The PX form Fe^{III}[Fe^{III}(CN)₆] possesses no surface or interior ionization in aqueous environments. Once the nanoparticles lose ionization, there remains no electrostatic binding to LPEI. The PX nanoparticles should be non-dispersable and hydrophobic, so they would not directly diffuse from the film surface into the aqueous electrolyte. Instead, film dissolution proceeds because PX nanoparticles do not compensate cationically ionized LPEI, which remains partially ionized at the electrolyte pH of 4. Thus when the film is polarized to oxidative potentials greater than ˜1.0 V, LPEI becomes charge—charge repulsed by adjacent LPEI chains, causing film desorption. Presumably, hydrophobic PX particles are physically carried away from the film by desorbing LPEI.

This controllable dissolution phenomenon was investigated further using a sheared cell in which electrochemistry was performed with a continuous electrolyte flow parallel to the film surface of approximately 31 s⁻¹. The sheared cell provides convective removal of desorbed LPEI and nanoparticles from the film surface, a method superior to that provided by the quiescent spectroelectrochemistry cell used to collect FIG. 10A that relies on gravity and diffusion. Films immersed into the sheared cell were switched for different numbers of cycles using the same waveform employed in FIG. 10A. Following this treatment, the thickness and UV-vis absorbance of each film was evaluated. Comparison was also made with a film that had been immersed in the shear cell for 25 min without polarization and with a film that had been switched 25 times between the PW and PB states. Thickness results are shown in the histogram FIG. 10B. Soaking or PW⇄PB cycling in the shear cell does not influence the LPEI/PB film thickness, but PB⇄PX cycling dramatically reduces the film thickness with an increasing number of cycles. Roughness did not vary significantly, increasing from 1.8 nm to 2.7 nm after 25 PB⇄PX cycles. By using the extinction coefficient previously determined in this work, the molar amount of PB could be estimated and a compositional profile of the PB⇄PX cycled films could be determined from UV-vis spectra. The 10 and 25 PB⇄PX cycle films exhibited lowered LPEI/PB molar repeat ratios of 1.14 and 0.53, respectively. These values indicate that LPEI within the film is removed more rapidly than PB, which confirms that the particle loss mechanism is initiated by LPEI desorption. The remaining particles are very weakly bound by a smaller amount of remaining LPEI; attempts to image the surface of partially dissolved films by tapping-mode AFM failed because the particles were moved by the tip, resulting in unacceptable skipping.

### Example 6

### Controlled Release of Heparin from PB/PEI Multilayers

Fifteen tetralayer films of LPEI/heparin/LPEI/PB was assembled using the techniques described in Example 2. Films were cycled between 0.2 V and 1.2 V at rates of 5, 10, 15, 20, 35, and 100 mV/s. The films degraded with nearly constant surface roughness (data not shown) on the scale of the thickness of a single layer, suggesting a top-down degradation mechanism. As shown in FIG. 11, the rate of drug release varies as the scan rate is changed, and the total amount of heparin released is dependent on the number of scan cycles.

### Example 7

### Measurement of Heparin Activity After Release from Redox-Active Multilayer Films

Heparin is a fact-acting anticoagulant that reversibly binds antithrombin III, catalyzing its inactivation of thrombin, factor Xa, and other serine proteases involved in blood coagulation. Thus, factor Xa activity scales inversely with heparin activity. An anti-factor Xa assay is used to compare the relative activity of heparin before encapsulation and after its release from multilayer films. Factor Xa activity is also used to measure the release rate of heparin from multilayer films.

### Example 8

### In Vitro Assay of Cellular and Systemic Toxicity of Thin Films

A conventional MTT cytotoxicity survey is used to determine the cellular toxicity of thin film degradation products. The MTT assay measures the effect of an added substance on cell growth and metabolism. Human cervical cancer cells (HeLa) are incubated with increasing concentrations of thin film degradation products in a a 96 well plate format. Following incubation, the media are replaced with fresh media and the cells are incubated for 72 hr. Cell viability is then measured by the emission of light at 570 nm, which reflects absorbance of a formazan crystal that is produced by healthy cells, and normalized to the value for healthy, untreated cells.

To determine any negative immunological effects of the thin films, activation of the complement system is measured. The complement system is a crucial part of the nonspecific immune response stimulated by the binding of an antibody to an antigen; as a result, a series of blood proteins are activated in a cascade of events that ultimately results in a number of undesired immunological defense mechanisms. To examine activation of the complement system by thin film degradation products, a modified form of the total hemolytic complement activity assay is used. The total hemolytic complement activity assay makes use of antibody sensitized sheep erythrocytes (SRBCs), which are combined with diluted serum containing increasing amounts of thin film degradation products (up to 1 mg/mL) and triethanolamine buffered saline (TBS) containing NaCl. Control absorbance of released hemoglobin is measured from reaction mixtures incubated with TBS and pure water, which represent 0% and 100% lysis, respectively. Finally, the percentage of complement activation (Y) is determined using the following formula (L.-C. Chang, H.-F. Lee, M.-J. Chung, V. C. Yang, Bioconjugate Chem. 2004, (ASAP Article, In Press)):

$$Y = ( 1 - ( \frac{{OD}_{541} - {OD}_{TBS , 541}}{{OD}_{Water , 541} - {OD}_{TBS , 541}} ) ) \times 100$$

In all experiments, results are compared with standard control polymeric biomaterials poly(ethylene glycol) (PEG) and poly (aspartic-co-glutamic acid) (PLGA), FDA-approved materials for which complement activation is well understood.

### REFERENCES

1. K. E. Uhrich, S. M. Cannizzaro, R. S. Langer, K. M. Shakesheff, “Polymeric systems for controlled drug release”, Chem. Rev. 1999, 99, 3181-3198.
2. K. Christensen, R. Larsson, H. Emanuelsson, G. Elgue, A. Larsson, “Heparin coating of the stent graft—effects on platelets, coagulation and complement activation”, Biomaterials 2001, 22, 349-355.
3. C. Hehrlein, A. Arab, C. Bode, “Drug-eluting stent: the “magic bullet” for prevention of restenosis?” Basic Res. Cardiol. 2002, 97, 417-423.
4. T. C. Poerner, K. K. Haase, B. Wiesinger, J. Wiskirchen, S. H. Duda, “Drug-coated stents”, Minimally Invasive Therapy & Allied Technologies 2002, 11, 185-192.
5. T. C. Poerner, T. Sueselbeck, A. Baumbach, M. Oberhoff, C. Herdeg, S. Wnendt, M. Borggrefe, W. Koenig, K. K. Haase, “Coronary stents: recent developments and perspectives”, Perfusion 2002, 15, 434.
6. G. Oliva, M. Espallargues, J. M. V. Pons, “Antiproliferative drug-eluting stents: Systematic review of the benefits and estimate of economic impact”, Revista Espanola De Cardiologia 2004, 57, 617-628.
7. J. T. Santini, A. C. Richards, R. Scheidt, M. J. Cima, R. Langer, “Microchips as controlled drug-delivery devices”, Angewandte Chemie-International Edition 2000, 39, 2397-2407.
8. D. A. LaVan, T. McGuire, R. Langer, “Small Scale Systems for in vivo drug delivery”, Nature Biotechnology 2003, 21, 1184-1191.
9. S. Z. Razzacki, P. K. Thwar, M. Yang, V. M. Ugaz, M. A. Burns, “Integrated Microsystems for controlled drug delivery”, Adv. Drug Deliv. Rev. 2004, 56, 185-198.
10. A. C. Richards Grayson, R. Scheidt Shawgo, Y. Li, M. J. Cima, “Electronic MEMS for triggered delivery”, Adv. Drug Deliv. Rev. 2004, 56, 173-184.
11. J. T. Santini, M. J. Cima, R. S. Langer, “Microchips for drug delivery”, Abstracts of Papers of the American Chemical Society 2000, 219, U34-U34.
12. G. Decher, J-D Hong, “Buildup of ultrathin multilayer films by a self-assembly process, 1) Consecutive Adsorption of Anionic and Cationic Bipolar Amphiphiles on Charged Surfaces”, Makromol. Chem., Macromol. Symp. 1991, 46, 321-327.
13. G. Decher, J-D Hong, “Buildup of Ultrathin Multilayer Films by a Self-Assembly Process: II. Consecutive Adsorption of Anionic and Cationic Bipolar Amphiphiles and Polyelectrolytes on Charged Surfaces”, Ber. Bunsenges, Phys. Chem. 1991, 95, 1430-1434.
14. G. Decher, B. Lehr, K. Lowack, Y. Lvov, J. Schmitt, J. Biosens. Bioelectron. 1994, 9, 677.
15. G. Decher, “Fuzzy Nanoassemblies: Toward Layered Polymeric Multicomposites”, Science 1997, 277, 1232.
16. G. Decher, M. Eckle, J. Schmitt, B. Struth, “Layer-by-layer assembled multicomposite films”, Curr. Op. Coll. & Int. Sci. 1998, 3, 32-39.
17. P. T. Hammond, “Form and Function in Multilayer Assembly: New Applications at the Nanoscale”, Adv. Mater. 2004, 16, 1271-1293.
18. D. M. DeLongchamp, P. T. Hammond, Chem. Mater. 2003, 15, 1165.
19. D. Ellis, M. Eckhoff, V. D. Neff, J. Phys. Chem. 1981, 85, 1225.
20. K. P. Rajan, V. D. Neff, .1. Phys. Chem. 1982, 86, 4361.
21. I. Moriguchi, H. Kamogawa, K. Hagiwara, Y. Teraoka, Chem. Lett. 2002, 310.
22. P. J. Peerce, A. J. Bard, J. Electroanal. Chem. 1980, 114, 89.
23. D. M. DeLongchamp, M. Kastantin, P. T. Hammond, Chem. Mater. 2003, 1 1575.
24. H. J. Buser, D. Schwarzenbach, W. Petter, A. Ludi, Inorg. Chem. 1977, 16, 2704.
25. M. B. Robin, Inorg Chem. 1962, 1, 337.
26. K. Itaya, K. Shibayama, H. Akahoshi, S. Toshima, J. Appl. Phys. 1982, 53, 804.
27. R. Vittal, M. Jayalakshmi, H. Gomathi, G. P. Rao, J. Electrochem. Soc. 1999, 146, 786.
28. M. K. Carpenter, R. S. Conell, J. Electrochem. Soc. 1990, 137, 2464.
29. M. A. Habib, S. P. Maheswari, J. Electrochem. Soc. 1992, 139, 2155.
30. E. A. R. Duck, M. A. Depaoli, M. Mastragostino, Adv. Mater. 1993, 5, 650.
31. M. A. Habib, S. P. Maheswari, M. K. Carpenter, J. Appl. Electrochem. 1991, 21, 203.
32. B. P. Jelle, G. Hagen, S. Nodland, Electrochim. Acta 1993, 38, 1497.
33. S. M. Chen, J. Electroanal. Chem. 2002, 521, 29.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A decomposable thin film comprising a plurality of alternating layers of net positive and negative charge, wherein at least a portion of the positive layers, the negative layers, or both, comprise a polyelectrolyte, wherein the layers are stable with respect to delamination at a first predetermined voltage, and wherein the thin film is not stable at a second predetermined voltage.

2. The decomposable thin film of claim 1, wherein the first predetermined voltage is no applied voltage.

3. The decomposable thin film of claim 1, wherein at least a portion of the layers 9 of net positive charge comprise a first polyelectrolyte that carries a positive charge at the first predetermined voltage.

4. The decomposable thin film of claim 3, wherein the first polyelectrolyte comprises one or more of linear polyethylene imine, branched polyethylene imine, polyallylamine HCl (PAH), polylysine, chitosan, poly(diallydimethylammonium chloride) (PDAC), polysaccharides, polymers of positively charged amino acids, polyaminoserinate, hyaluronan, poly(L-lactide-co-L-lysine), poly(serine ester), poly(4-hydroxy-L-proline ester), poly[α-(4-aminobutyl)-L-glycolic acid], and co-polymers, mixtures, and adducts of any of the above.

5. The decomposable thin film of claim 3, wherein the first polyelectrolyte comprises a polymer having ionizable groups selected from amine, quaternary ammonium, quaternary phosphonium, and any combination of the above, wherein the ionizable groups are disposed in groups pendant from a backbone of the polymer, attached to the backbone directly, or incorporated in the backbone of the polymer.

6. The decomposable thin film of claim 1, wherein at least a portion of the layers of net negative charge comprise a second polyelectrolyte that carries a negative charge at the first predetermined voltage.

7. The decomposable thin film of claim 6, wherein the second polyelectrolyte comprises polymalic acid, hyaluronic acid, polymers of negatively charged and acidic amino acids, polynucleotides, poly beta amino esters, and co-polymers, mixtures, and adducts of any of the above.

8. The decomposable thin film of claim 6, wherein the second polyelectrolyte comprises a polymer having ionizable groups selected from carboxylate, sulfonate, sulphate, phosphate, nitrate, and combinations of the above, wherein the ionizable groups are disposed in groups pendant from a backbone of the polymer, attached to the backbone directly, or incorporated in the backbone of the polymer.

9. The decomposable thin film of claim 1, wherein at least a portion of the layers comprise a conducting polymer or a redox polymer.

10. The decomposable thin film of claim 1, wherein at least a portion of the layers comprise a dendrimer.

11. The decomposable thin film of claim 1, wherein at least a portion of the layers of net negative charge comprise Prussian Blue.

12. The decomposable thin film of claim 1, wherein at least a portion of the layers comprise a first active agent.

13. The method of claim 12, wherein the first active agent comprises a drug, a protein, an oligopeptide, or a polynucleotide.

14. The method of claim 13, wherein the first active agent is encapsulated by a micelle, a dendrimer, or a nanoparticle.

15. The decomposable thin film of claim 12, wherein the first active agent is retained on the polyelectrolyte in the positive or negative layers by covalent or non-covalent interactions.

16. The decomposable thin film of claim 12, wherein the concentration of the first active agent varies among the layers.

17. The decomposable thin film of claim 12, wherein the concentration of the first active agent describes a gradient from a top layer of the thin film to a bottom layer of the thin film.

18. The decomposable thin film of claim 12, wherein the thin film comprises alternating pluralities of layers that do and do not contain the first active agent.

19. The decomposable thin film of claim 12, wherein at least a portion of the layers comprise a second active agent.

20. The decomposable thin film of claim 19, wherein at least a portion of the layers comprise both the first active agent and the second active agent.

21. The decomposable thin film of claim 19, wherein at least a portion of the layers include either the first active agent or the second active agent.

22. The decomposable thin film of claim 19, comprising a plurality of substantially active agent-free layers interspersed between layers comprising the first active agent and layers comprising the second active agent.

23. The decomposable thin film of claim 1, wherein the thin film is disposed on a substrate having a shape selected from particles, tube, sphere, strand, coiled strand, and capillary network, sponge, cone, portion of cone, rod, strand, coiled strand, capillary network, film, fiber, mesh, sheet, and threaded cylinder.

24. The decomposable thin film of claim 1, wherein the thin film is disposed on a substrate having a texture having a size scale between about 100 nm and about 500 nm.

25. The decomposable thin film of claim 1, wherein the thin film is deposited on a substrate having a texture selected from bumps, grooves, raised ridges, teeth, threads, wedges, cylinders, pyramids, blocks, dimples, holes, and grids.

26. The decomposable thin film of claim 1, wherein the thin film is deposited on a substrate comprising a metal, ceramic, polymer, or semiconductor material.

27. The decomposable thin film of claim 26, wherein the thin film includes a buffer comprising a plurality of polyelectrolyte bilayers that are stable with respect to an applied voltage, wherein said buffer is disposed between the plurality of alternating layers and a substrate.

28. The decomposable thin film of claim 1, wherein the layers of the thin film delaminate sequentially in response to the second predetermined voltage.

29. The decomposable thin film of claim 1, wherein at least a portion of the film is organized in tetralayer heterostructures comprising first and second layers having a first charge and the same composition and third and fourth layers interspersed with the first and second layers and having a second charge, wherein the third layer comprises an active agent having a predetermined physiological target and the fourth layer comprises a material that is inactive with respect to the predetermined target.

30. The decomposable thin film of claim 1, wherein the film is from 1 to 10 nm thick.

31. The decomposable thin film of claim 1, wherein the film is from 10 to 100 nm thick.

32. The decomposable thin film of claim 1, wherein the film is from 100 to 1000 nm thick.

33. The decomposable thin film of claim 1, wherein the film is from 1000 to 5000 nm thick.

34. The decomposable thin film of claim 1, wherein the film is from 5000 to 10,000 nm thick.

35. A drug delivery device, comprising:
a support; and
at least one decomposable thin film according to claim 1 disposed on the support.

36. The drug delivery device of claim 35, further comprising a first electrode and a second electrode disposed on opposing sides of the decomposable thin film.

37. The drug delivery device of claim 35, wherein the first and second electrodes may be in electrical communication with a microprocessor that controls when a voltage is applied across the first and second electrodes.

38. A method of generating a three-dimensional structure on a surface, comprising:
providing a charged region on the surface; and
assembling a plurality of layers of alternating charge on the surface,
wherein at least a portion of the layers exhibit a change in net charge upon a change in an applied voltage.

39. The method of claim 38, wherein the surface has a contour selected from particles, tube, sphere, strand, coiled strand, and capillary network, sponge, cone, portion of cone, rod, strand, coiled strand, capillary network, film, fiber, mesh, sheet, and threaded cylinder.

40. The method of claim 39, wherein assembling a plurality of layers comprises immersing at least a portion of the surface in alternating solutions containing layer-forming materials of opposite charge.

41. The method of claim 38, wherein assembling the plurality of layers comprises assembling a plurality of discrete pluralities of layers on the surface.

42. The method of claim 41, wherein the discrete pluralities of layers do not all have the same composition.

43. The method of claim 38, wherein assembling comprises one or more of spray coating, ink-jet printing, brush coating, roll coating, spin coating, soft lithography, microcontact printing, multilayer transfer printing, layer-by-layer deposition, and roll-to-roll coating.

44. A method of controllably releasing a material from a thin film comprising a plurality of layers of alternating charge in which the material is disposed, comprising:
changing an applied voltage from a first value to a second value at a predetermined frequency, wherein at least a portion of the layers exhibit a reduced net charge at the second value.

45. The method of claim 44, wherein either the first value or the second value is 0 V.

46. A method of controllably releasing material from a plurality of discrete thin films disposed on a surface, each thin film comprising a plurality of layers of alternating charge, comprising:
applying a first predetermined voltage at a first predetermined frequency to a first predetermined member of the plurality of thin films, wherein at least a portion of the layers in the first predetermined member exhibit a reduced net charge at the first predetermined voltage.

47. The method of claim 46, further comprising applying a second predetermined voltage at a second predetermined frequency to a second predetermined member of the plurality of thin films at a predetermined time interval following applying the first predetermined voltage, wherein at least a portion of the layers in the second predetermined member exhibit a reduced net charge at the second predetermined voltage.

48. The method of claim 47, wherein the first predetermined voltage and the second predetermined voltage are applied to both the first predetermined member and the second predetermined member.
